(19) United States
(12) Patent Application Publication
(10) Pub. No.: US 2017/0030942 A1
(43) Pub. Date: Feb. 2, 2017

(54) SYSTEMS AND METHODS FOR DETERMINING VELOCITY AND FLUX OF A GAS

(71) Applicant: LI-COR, INC., Lincoln, NE (US)

(72) Inventor: Tyler Anderson, Lincoln, NE (US)

(73) Assignee: LI-COR, INC., Lincoln, NE (US)

(21) Appl. No.: 15/290,837

(22) Filed: Oct. 11, 2016

Related U.S. Application Data
(62) Division of application No. 14/830,521, filed on Aug. 19, 2015.
(60) Provisional application No. 62/039,304, filed on Aug. 19, 2014.

(51) Int. Cl. G01P 5/18; G01N 33/00

(52) U.S. Cl. CPC G01P 5/18; G01N 33/0031

(57) ABSTRACT

Systems and methods for determining gas velocity based on phase differences of signals from two or more interaction paths in a gas analyzer system. A laser source, which can provide access to an absorption gas line, is expanded, or is split into two or more beams. These beams can be used to create two (or more) parallel sampling paths separated by a known distance. Gas travelling in the plane of the two beams of light will pass through the optical paths at two (or more) different times creating very similar signals that will be out of phase with each other. The amount of phase difference will be inversely proportional to the velocity of the gas.

### CROSS-REFERENCE TO RELATED APPLICATIONS

This Application is a divisional of copending U.S. patent application Ser. No. 14/830,521, filed Aug. 19, 2015, which claims priority to U.S. Provisional Patent Application No. 62/039,304, filed Aug. 19, 2014. The disclosures of these applications are hereby incorporated by reference.

### BACKGROUND

The present disclosure relates to gas analysis and flux measurements, and more particularly to gas velocity and flux measurements based on phase differences of signals along two or more separate detection paths.

The increasing concentrations of carbon dioxide and other traces gases (e.g. H₂O, CH₄, N₂O, NH₃, etc.) in the atmosphere and the resulting greenhouse effect and climate change have become important topics for scientific research. For example, in order to understand the global carbon balance, it is necessary to determine the rate at which carbon dioxide and energy exchanges between the atmosphere and terrestrial and oceanic ecosystems. The air within a few hundred meters above the earth's surface is mostly turbulent, so that turbulent structures (vortices of variable sizes) called “eddies” are responsible for the vertical transport of most of the gases, including carbon dioxide and water vapor, and also heat and momentum between the surface and the atmosphere. The rates of such transport can be calculated from simultaneous, high-frequency measurements of the vertical component of wind speed, the concentrations of carbon dioxide and water vapor, and the air temperature.

Currently, there are various methods for computing turbulent gas flux rates, usually performed from towers, and other platforms. Typically, air velocity is performed using an anemometer, such as a sonic anemometer, or other air velocity measuring device, and gas concentration is determined using a gas analyzer instrument, such as an IRGA-based gas analyzer.

It is desirable to provide improved systems and methods for measuring velocity of a gas, and also improved systems and methods for measuring gas flux.

### SUMMARY

The present disclosure provides systems and methods for determining gas velocity based on phase differences of signals from two or more interaction paths in a gas analyzer system, and also for determining gas flux measurements.

According to an embodiment, a device for measuring the velocity of a gas is provided. The device typically includes a laser source that emits laser light, and optical elements configured to split the emitted laser light into first and second co-linear beam paths ending at first and second detectors, respectively, the first and second beam paths separated by a first distance along a first direction perpendicular to the first and second beam paths. The device also typically includes the first detector that detects laser light from the first beam path and outputs a first signal representing a first optical power in the first beam path, and the second detector that detects laser light from the second beam path and outputs a second signal representing a second optical power in the second beam path. The device also typically includes an intelligence module, such as one or more processors, coupled with the first and second detectors and configured to receive the first and second signals and, based on the first distance and on the first and second signals, compute a velocity of the gas along the first direction.

In certain aspects, the intelligence module is further configured to compute a concentration of the gas and determine a flux of the gas. In certain aspects, the gas is water vapor, or the gas includes at least one of H₂O, CO₂, CH₄, N₂O, NH₃, or isotopes of CO₂ and/or H₂O.

According to another embodiment, a method of measuring gas velocity is provided. The method typically includes splitting a beam of laser light into first and second co-linear beam paths, where the first and second beam paths are separated by a first distance along a first direction and ending at first and second detectors, respectively. The method also typically includes detecting laser light from the first beam path with the first detector and outputting a first signal representing a first optical power in the first beam path, and detecting laser light from the second beam path with the second detector and outputting a second signal representing a second optical power in the second beam path. The method further typically includes computing, e.g., using an intelligence module such as a processor, and based on the first distance and on the first and second signals, a velocity of the gas along the first direction.

In certain aspects, the method further includes computing a concentration of the gas and determining a flux of the gas. In certain aspects, the gas is water vapor, or the gas includes at least one of H₂O, CO₂, CH₄, N₂O, NH₃, or isotopes of CO₂ and/or H₂O.

According to yet another embodiment, a device for measuring the velocity of a gas is provided. The device typically includes a laser source that emits laser light having a first diameter, and optical elements configured to expand the emitted laser light into a beam having a second diameter larger than the first diameter. The device also typically includes a detector element that detects laser light from the beam and outputs signals representing optical power in each of at least two regions of the beam, and an intelligence module, coupled with the detector element and configured to receive the signals and, based on distances between the at least two regions and on the output signals, compute a velocity of the gas along at least a first direction perpendicular to the beam.

In certain aspects, the detector element detects laser light from the beam and outputs signals representing densities of the gas in each of at three equal regions of the beam, and wherein the intelligence module, based on distances between the at least three regions, compute a velocity of the gas along the first direction and a second direction, the second direction being perpendicular to the beam and to the first direction. In certain aspects, the detector element includes a quadrant detector that detects in four different regions. In certain aspects, the detector element comprises a separate detector for each of the at least two regions being detected. In certain aspects, the gas includes at least one of H₂O, CO₂, CH₄, N₂O, NH₃, or isotopes of CO₂ and/or H₂O.

According to yet another embodiment, a device for measuring the velocity of a gas is provided. The device typically includes a laser source that emits laser light having a first diameter, and optical elements configured to expand the emitted laser light into a beam having a second diameter larger than the first diameter. The device also typically includes a beamsplitter element that splits the beam into first and second beams, the first beam and the second beam being substantially perpendicular to each other, and a first detector element that detects laser light from the first beam and outputs first signals representing optical power in each of at least three equal regions of the first beam, and a second detector element that detects laser light from the second beam and outputs second signals representing optical power in each of at least three equal regions of the second beam. The device further typically includes an intelligence module, coupled with the first and second detector elements and configured to receive the first and second signals and, based on distances between the at least three regions in each of the first and second beams and on the first and second signals, compute a velocity of the gas along three orthogonal directions.

In certain aspects, the detector element includes a quadrant detector that detects in four different regions. In certain aspects, the detector element comprises a separate detector for each of the at least two regions being detected. In certain aspects, the gas includes at least one of H₂O, CO₂, CH₄, N₂O, NH₃, or isotopes of CO₂ and/or H₂O.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates a system configuration including a beamsplitter and a mirror according to an embodiment.

FIG. 2 illustrates a system configuration including a single beam imaged onto a quadrant detector for 2D velocity information according to an embodiment.

FIG. 3 illustrates a system configuration including 2 quadrant detectors and a beam splitter to obtain 3D velocity information according to an embodiment.

FIG. 4 illustrates concentration time series from side-by-side optical paths.

FIG. 5 illustrates differences calculated between signals.

FIG. 6 shows raw absorption values from 2 point measurements for an experimental setup as shown in FIG. 1.

FIG. 7 shows a cross correlation (or other phase measurement technique) is calculated for the 2 data streams.

FIG. 8 shows plots of phase difference v. flow velocity, and calculated flow v. flow velocity.

FIG. 9 illustrates a method of measuring gas velocity according to an embodiment.

### DETAILED DESCRIPTION

The present disclosure provides systems and methods for determining gas velocity based on phase differences of signals from two or more interaction paths in a gas analyzer system, and also for determining gas flux measurements.

In certain embodiments, the velocity of a gas, such as water vapor, can be determined by making two gas measurements, using interaction paths spatially separated by a known distance, and measuring the phase difference between signals representative of gas detection from those interaction paths. A laser source, which can provide access to an absorption gas line, is expanded, or is split into two or more beams. These beams can be used to create the two (or more) parallel sampling paths. Gas travelling in the plane of the two beams of light will pass through the optical paths at two (or more) different times creating very similar signals that will be out of phase with each other. The amount of phase difference will be inversely proportional to the velocity of the gas. In certain embodiments, the beams create two perpendicular paths for 3-dimensional velocity measurements (e.g., along three perpendicular axes).

Certain embodiments advantageously combine a gas concentration measurement with a velocity measurement to measure the flux of the gas with a single instrument and reduce resource consumption, e.g., data management and computation resources. Gas density and velocity measurements are advantageously combined into a single device. Flux computations are advantageously simplified by co-located velocity and concentration measurements, components for the measurement are simple and cost effective (e.g., likely cost less than the cost of a gas analyzer and a sonic anemometer combined), and one instrument becomes easier for a customer to setup and can prevent setup errors that result in bad data or data loss.

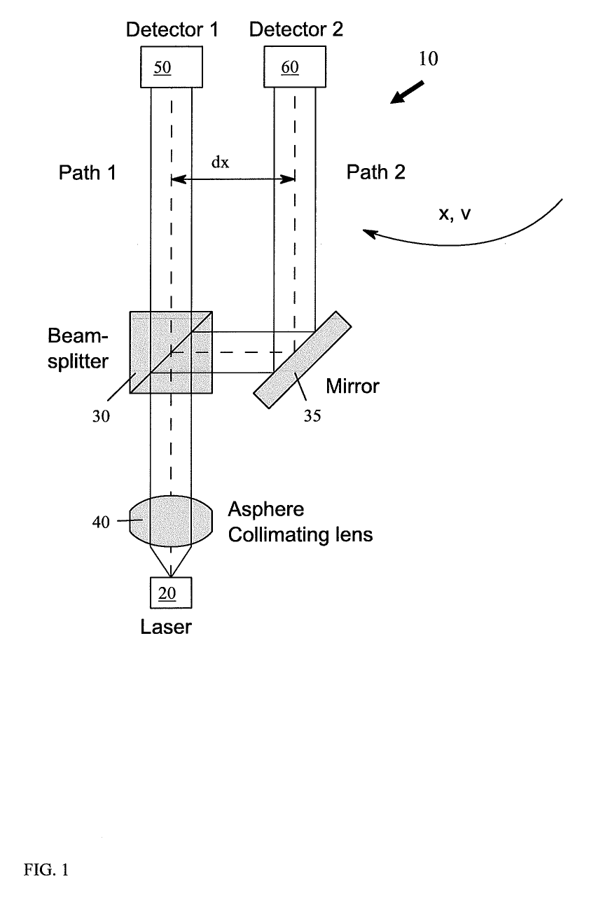

FIG. 1 illustrates components of a gas analysis device 10 according to an embodiment. The setup in FIG. 1 allows for 1-dimensional (1D) velocity measurement using a light source output split into two paths separated by (center-to-center) distance dₓ as shown in FIG. 1. For example, as shown in FIG. 1, a device 10 for measuring the velocity of a gas includes a laser source 20 that emits laser light, optical elements configured to split the emitted laser light into first and second co-linear beam paths (path 1 and path 2) ending at first detector 50 and second detector 60, respectively, with the first and second beam paths separated by a first distance dₓ along a first direction (arbitrary, x-direction in FIG. 1) perpendicular to the first and second beam paths. The optical elements as shown in FIG. 1 include a beamsplitter 30, a reflective element or mirror 35 and a beam shaping optics 40. Beam shaping optics 40 is included where it is desirable to expand the beam size and/or collimate the beam as is well known. This and other device embodiments include a housing structure (not shown) to provide structural stability and to hold the various device components in appropriate relation to each other.

First detector 50 detects laser light from the first beam path and outputs a first signal representing a first optical power in the first beam path, and second detector 60 detects laser light from the second beam path and outputs a second signal representing a second optical power in the second beam path. The raw output of the detectors is optical power. The raw output may be used to obtain an absorption value of a gas in the interaction path of the beam (e.g., a gas or gas component that absorbs at the laser frequency propagating in the interaction path) or optical density of the gas. Temperature of the gas and the path length propagation of the beam may be used to compute density. A technique called balanced ratiometric detection (BRD) may be used for comparing differences between two raw signals.

There are multiple ways of analyzing the optical signal to provide different properties of the gas. All of these properties are based on absorptance, (I/I₀), or the ratio of the attenuated intensity of laser light to the available laser light. In one embodiment, for example, gas density may be determined based on the optical intensity value, where density means “number density” of the gas or moles/m̂3. In certain aspects, for example, absorptance (I/I₀), absorbance A=log(I/I₀), number density (rho=A/(S*L)), concentration (c=A*P/(S*L*R*T)) may be determined and used for further computations, where S=line strength, L=path length of the laser beam through the sampled gas, R=ideal gas constant, T=gas temperature, and P=gas pressure.

In certain embodiments, temperature and pressure sensors are provided to measure temperature and pressure of the gas for full concentration measurements of the gas. For example, a single temperature sensor and/or a single pressure sensor may be used, or multiple temperature sensors may be used (e.g., to determine an average T) or multiple pressure sensors may be used (e.g., to determine an average P). The device 10 also includes an intelligence module (not shown), coupled with the first and second detectors and configured to receive the first and second signals and, based on the first distance, dx, compute a velocity of the gas along the first direction. The intelligence module may include one or more processors and one or more memory elements for storing collected data and/or code to control operation of device components.

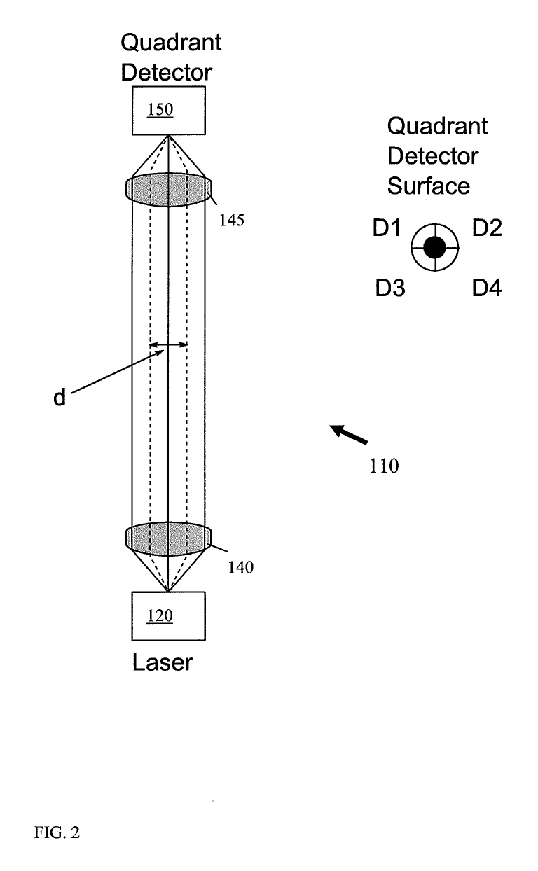

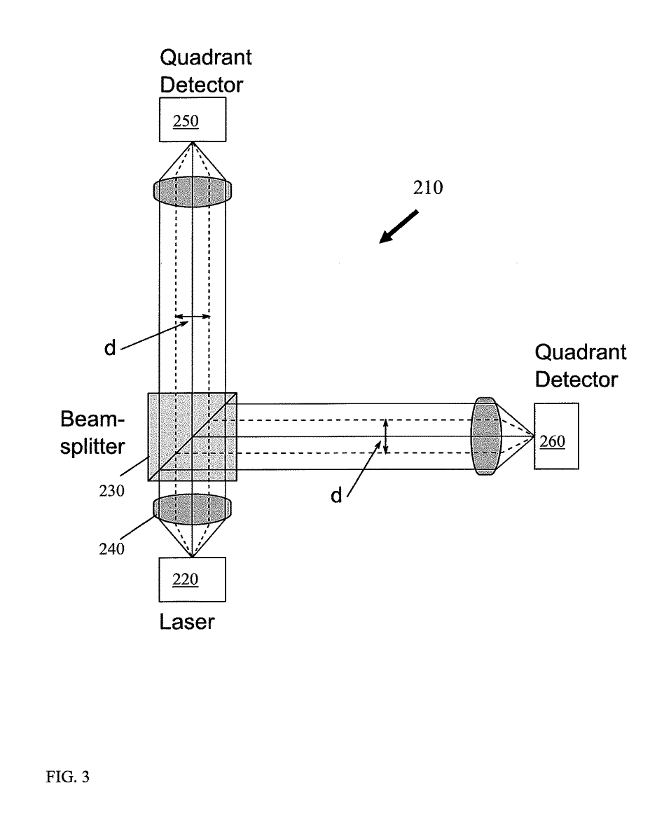

Other potential embodiments are shown in FIGS. 2 and 3. The embodiment shown in FIG. 2 enables 2D velocity measurements and the embodiment of FIG. 3 enables 3D velocity measurements.

FIG. 2 illustrates components of a gas analysis device 110 for measuring the flux of a gas according to an embodiment. Device 110 includes a laser source 120 that emits laser light having a first diameter, optical elements (e.g., beam shaping optics 140) configured to expand the emitted laser light into a beam having a second diameter larger than the first diameter, and travelling along a detection path. Detector element 150 detects laser light from the beam and outputs signals representing optical power in each of at least two regions of the beam (4 regions shown in FIG. 2). The at least two regions of the beam may be equal in size. An intelligence module (not shown), is coupled with the detector element and is configured to receive the signals output by the detector element 150 and, based on a distance (e.g., d in FIG. 2) between the at least two regions, compute a velocity of the gas along at least a first direction perpendicular to the beam.

In certain aspects, the detector element 150 detects laser light from the beam and outputs signals representing optical power in each of at three regions of the beam (e.g., 4 regions, D1, D2, D3 and D4, as shown in FIG. 2). The at least three regions of the beam may be equal in size. The intelligence module (not shown), based on distances between the at least three regions, compute a velocity of the gas along first and second directions, where both the first and the second directions are perpendicular to the beam, and the first direction is perpendicular to the second direction. In certain aspects, the detector element comprises a quadrant detector that detects in four different regions. The four regions of the beam may be equal in size. The intelligence module (not shown), based on distances between the four regions, computes a velocity of the gas along first and second directions, where both the first and the second directions are perpendicular to the beam, and the first direction is perpendicular to the second direction. In certain aspects, a grid array provides an orthogonal basis set of velocity vectors that can construct any other velocity vector. The detection array only has to be partially orthogonal but not strictly orthogonal, however (e.g., the dot product of two detection directions does not have to equal 0, but the cross product of two detection directions cannot equal 0).

In certain aspects, the detector element 150 includes a separate detector element for each of the regions being detected. In this manner, light in the laser beam is effectively partitioned at the detector(s).

FIG. 3 illustrates components of a gas analysis device 210 for measuring the flux of a gas according to an embodiment. Device 210 includes a laser source 220 that emits laser light having a first diameter and optical elements (e.g., beam shaping optics 240) configured to expand the emitted laser light into a beam having a second diameter larger than the first diameter, and a beamsplitter element 230 that splits the beam into first and second beams, the first beam and the second beam being substantially perpendicular to each other as shown. For example, the beam paths may be exactly perpendicular) (90°) (e.g., an orthogonal basis set), or they may vary from perpendicular by a few degrees (e.g., a partially orthogonal basis set).

The device 210 also includes a first detector element 250 that detects laser light from the first beam and outputs first signals representing optical power in each of at least two regions of the first beam, and a second detector element 260 that detects laser light from the second beam and outputs second signals representing optical power in each of at least two regions of the second beam. An intelligence module (not shown) is coupled with the first and second detector elements and is configured to receive the first and second signals. Based on distances between the regions in each of the first and second beams, the intelligence module computes a velocity of the gas along at least two orthogonal directions. The intelligence module computes gas density values from the raw optical power values, and uses the density values to compute phase difference. In certain aspects, the detector element 250 and the detector element 260 each detect light in at least three different regions of the respective beam, whereby the intelligence module computes a velocity of the gas along three orthogonal directions. In certain aspects, the first and second detector elements each comprise a quadrant detector that detects in four different regions. In certain aspects, the first and second detector elements each comprise a separate detector element for each of the two regions, or three regions or four regions being detected. It should be appreciated that one of detector element 250 or detector element 260 may detect a different number of regions of the beam than the other detector element. It should also be appreciated that a detector may detect more than four regions.

In certain aspects, the laser source is fiber coupled. This is advantageous as much of the system may be located away from the air sampling path and a designer gains freedom in how to configure the orientation of orthogonal sampling paths.

How or if the light gets split is unimportant. Other geometries better suited for splitting the beam could also be used. A simple dielectric coating on a surface of an optical element (e.g., beamsplitter or mirror element) could be designed to reflect and transmit 50% of the light or any proportion of light that would be better suited for the measurement. In certain aspects, two separate sources can be used, one for each beam path. The separate sources may operate with the same or with a different output frequency.

Splitting the wave front of a single source, however, keeps optical noise within the system common mode (up to the point that the light is split). The distance between the optical paths should be short enough to prevent gas diffusion from corrupting signals, but long enough to account for the finite modulation speed of sources available. For example, the distance between co-linear beam paths should be on the order of 1 cm to about 10 cm but can be larger or smaller. In certain aspects, the length of the beam paths (e.g., the length of the optical path within which the gas interacts with the laser beam) is on the order of 2.5 cm to 13 cm or 25 cm or more. The velocity of the gas will add a phase delay between the two signals being detected and analyzed. That delay will be inversely proportional to the velocity of the gas.

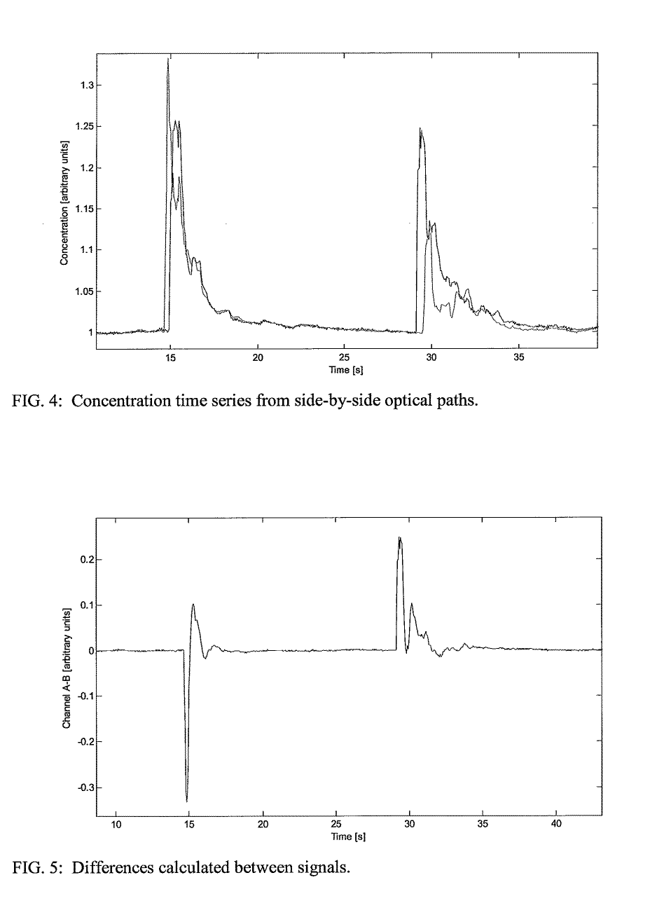

A configuration as shown in FIG. 1 was tested in the lab, and results are shown in FIGS. 4 and 5: FIG. 4 illustrates concentration time series from side-by-side optical path, and FIG. 5 illustrates differences calculated between signals. An 1854 nm laser, which can provide a 10% absorption signal for water with a few centimeters of interaction path, was used. The optical paths were separated by approximately 5 cm and the sampling rate was 20 Hz. Maximum resolvable velocities with this particular test setup are on the order of 1 m/s (20 Hz*0.5 m). Blowing air (with water vapor) through this setup provides a large enough signal with velocity to detect a concentration change and phase difference between channels.

Blowing air through the test setup of FIG. 1 from one direction produces a density or concentration pulse. As shown in FIGS. 4 and 5, the green data set detects the pulse first and leads the blue. However, in the second pulse (which was introduced from the opposite side of the test setup) the blue pulse leads the green. This can better be seen by subtracting one signal from the other (amplitude is related to phase difference; shape is related to the time derivative of the signal).

### Detectors and Sources

Water vapor absorption signals at 1392 nm and 1854 nm can easily reach 10 to 20% with just a few centimeters of path length. Direct absorption techniques can easily be used to measure absorption to obtain water concentrations. The 1392 nm bands are also within standard InGaAs detector spectral sensitivities. The 1854 nm bands require slightly more exotic extended InGaAs detectors. Other detectors may be used, such as InSb detectors, silicon-based detectors and other types.

DFB and VCSEL lasers both exist for each of these wavelengths bands. Shorter wavelengths will typically be much cheaper as well as DFB lasers. The disadvantage of DFBs is the speed of wavelength tuning is limited to 100 s of Hz. VCSELs, albeit more expensive, have much faster wavelength tuning, on the order of 10 s to 100 s of kHz.

The maximum velocity resolvable can be calculated from the modulation speed and the separation distance of the optical paths. For example, to resolve a 20 m/s wind speed, a 1 cm path separation would require a (20 m/s)/(0.01 m)=2000 Hz modulation frequency. DFB lasers fall outside of this range, but could plausibly make the measurement with a 10 to 20 cm path separation. Diffusion or small eddies at this point, however, may complicate the measurement and limit measurements to certain atmospheric conditions.

The device embodiments using a quadrant detector create a 2×2 “image” of the gas concentration and correlate these images in time to determine where and how fast gas flows are moving.

Various embodiments provide systems and methods for measuring velocities and densities of gasses such as H₂O, CO₂, CH₄, N₂O, NH₃, isotopes of CO₂ and H₂O, etc. For example, one particular application includes measuring evapotranspiration (ET) from a field. Where sufficient signal exists to measure isotopes of water or hydrogen, the components of ET (evaporation and transpiration) can be separated as both of these components have a different isotopic signature. The systems and methods are particularly useful in turbulent air structures. For example, in certain embodiments, the systems and methods advantageously sample and measure gas density, temperature and pressure at high speed and at high bandwidth, and allow for eddy covariance calculations, concentration calculations and calculation of dry mole fraction, or mixing ratio (or other similar units) of gas components. See, e.g., U.S. Pat. Nos. 8,433,525 and 7,953,558, which are hereby incorporated by reference, and which illustrate additional components and aspects of gas analysis systems for which the teachings provided herein are useful, including aspects of eddy covariance measurements.

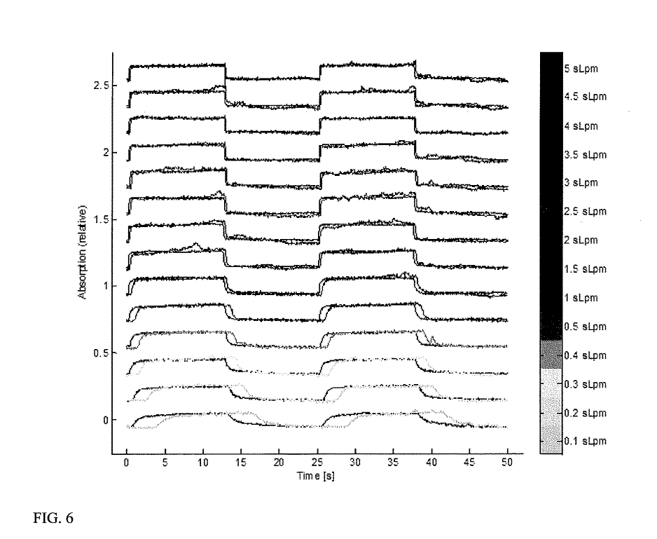

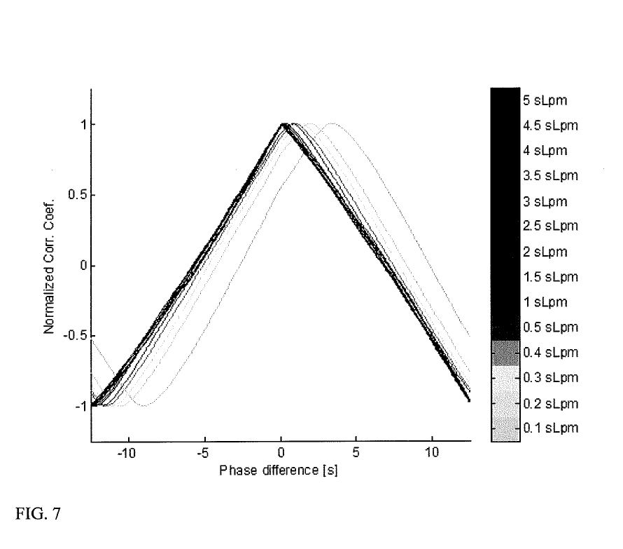

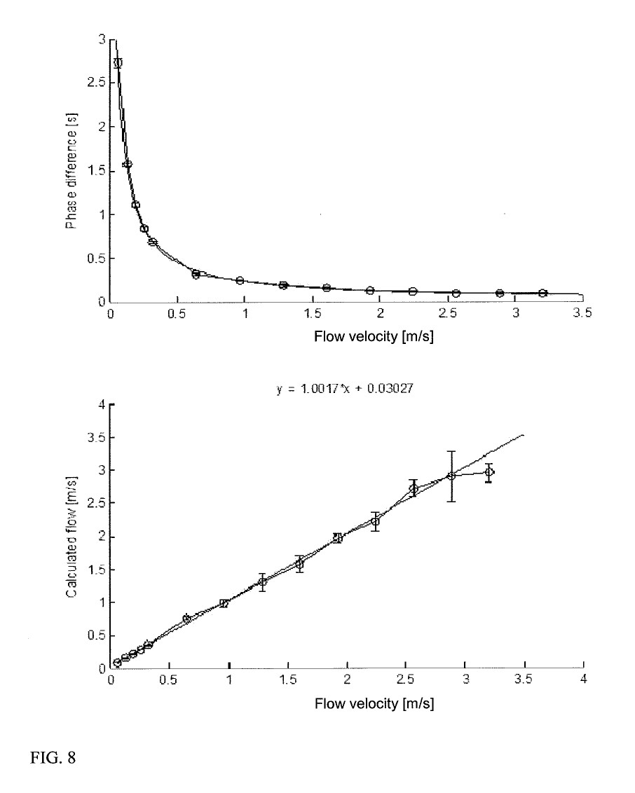

FIG. 6 shows raw absorption values from 2 point measurements for an experimental setup as shown in FIG. 1. As the flow rate of the gas stream increases, the phase shift between the 2 data streams becomes smaller. FIG. 7 shows a cross correlation (or other phase measurement technique) is calculated for the 2 data streams. This is the product of the 2 data streams as 1 dataset is shifted past the other. Maximum values occur when the data is shifted back in phase. The maximum value of the cross correlation function gives the time delay between the 2 datasets. The distance between the sensors divided by the calculated time of flight gives the velocity of the gas. FIG. 8 shows plots of phase difference v. flow velocity and calculated flow v. flow velocity. The maximum value of the cross correlation function (FIG. 7) occurs at a location along the x-axis equal to the phase difference or time of flight difference between the 2 data streams. The known sensor separation is divided by the phase difference or the time of flight (top plot of FIG. 8) to compute the gas flow velocity (bottom plot of FIG. 8). A one-to-one correspondence between the computed flow velocity and the known flow velocity supplied to the device depicted in FIG. 1 are obtained in the bottom plot of FIG. 8.

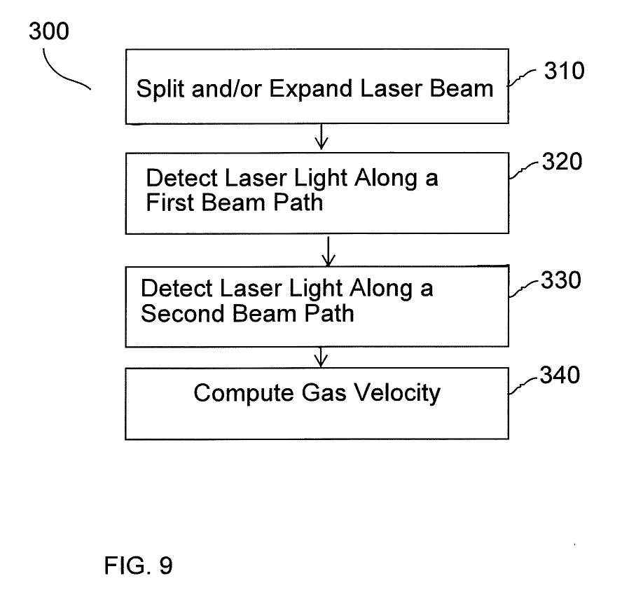

FIG. 9 illustrates a method 300 of measuring gas velocity according to an embodiment. In step 310, a beam of laser light is split into first and second beam paths, the first and second beam paths separated by a first distance along a first direction and ending at first and second detectors, respectively. In one embodiment, the beam paths are collinear, and in another embodiment the beam paths are perpendicular, or have vector components perpendicular to each other. In one embodiment, splitting the laser beam is accomplished using a beamsplitter element and other optical components, e.g., a mirror element, as is done in one embodiment as shown in, and discussed above with reference to, FIG. 1. Alternatively, in another embodiment, splitting the laser light may include expanding the output laser beam using a beam expanding element so that two (or more) regions of the beam path are separably detected as is discussed above with reference to FIG. 2. In step 320, laser light from the first beam path is detected with the first detector and a first signal representing a first density of a gas in the first beam path is output. In step 330, laser light from the second beam path is detected with the second detector and a second signal representing a second density of the gas in the second beam path is output. It should be appreciated that the detections in steps 320 and 330 are performed simultaneously, although the output of signals need not be performed simultaneously, so long as corresponding output signals are time correlated. In step 340, a velocity of the gas along the first direction is computed based on the first distance and on the first and second signals. Input (received) data and/or results data may be stored for later use, output to another system for further processing, and/or displayed on a display device. Also, a gas concentration can be determined and output and/or a gas flux can be determined and output based on the gas velocity measurement and other parameters, such as a gas concentration measurement, a temperature measurement, a pressure measurement, etc.

It should be appreciated that the gas analysis processes described herein may be implemented in computer code running on an intelligence module, e.g., one or more processors of a computer system. The code includes instructions for controlling a processor to implement various aspects and steps of the gas analysis processes. The code is typically stored on a non-transitory memory element such as a hard disk, RAM or portable medium such as a CD, DVD, etc. Similarly, the processes may be implemented in a gas analyzer instrument including an intelligence module, typically having one or more processors, executing instructions stored in a memory element coupled to the processor(s). The intelligence module may be part of the gas analyzer, or part of a separate system directly or indirectly coupled with the gas analyzer. Code including such instructions may be downloaded to the system or gas analyzer memory unit over a network connection or direct connection to a code source or using a portable, non-transitory computer-readable or processor-readable medium as is well known. It should also be understood that an intelligence module may be configured to merely collect and store data and that the collected data may be transmitted to, sent to, or otherwise provided to a separate system that implements the data processing and computations described herein. Also, an intelligence module is typically adapted to provide relevant output data, such as input (measurement) data, processed data, result data, etc. and visual and/or graphical representations of such data, e.g., to a display device for display to a user.

One skilled in the art should appreciate that the processes of the present invention can be coded using a variety of programming languages such as C, C++, C#, Fortran, VisualBasic, etc., as well as applications such as Mathematica® which provide pre-packaged routines, functions and procedures useful for data visualization and analysis. Another example of the latter is MATLAB®.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed embodiments and does not pose a limitation on the scope of the embodiments unless otherwise claimed. No language in the specification should be construed as indicating any non-claimed element as essential to the practice of the embodiments of the disclosure.

Certain embodiments of this invention are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1-7. (canceled)

8. A device for measuring the flux of a gas, comprising:
a laser source that emits laser light having a first diameter;
optical elements configured to expand the emitted laser light into a beam having a second diameter larger than the first diameter;
a detector element that detects laser light from the beam and outputs signals representing optical power in each of at least two regions of the beam; and
an intelligence module, coupled with the detector element and configured to receive the signals and, based on the signals and on a distance between the at least two regions, compute a velocity of the gas along at least a first direction perpendicular to the beam.

9. The device of claim 8, wherein the detector element detects laser light from the beam and outputs signals representing densities of a gas in each of at least three equal regions of the beam, and wherein the intelligence module is configured to, based on distances between the at least three regions, compute a velocity of the gas along the first direction and a second direction, the second direction being perpendicular to the beam and to the first direction.

10. The device of claim 8, wherein the detector element comprises a quadrant detector that detects in four different regions.

11. The device of claim 8, wherein the detector element comprises a separate detector for each of the at least two regions being detected.

12. The device according to claim 8, wherein the gas includes at least one of H₂O, CO₂, CH₄, N₂O, NH₃, or isotopes of CO₂ and/or H₂O.

13. A device for measuring the velocity of a gas, comprising:
a laser source that emits laser light having a first diameter;
optical elements configured to expand the emitted laser light into a beam having a second diameter larger than the first diameter;
a beamsplitter element that splits the beam into first and second beams, the first beam and the second beam being substantially perpendicular to each other;
a first detector element that detects laser light from the first beam and outputs first signals representing optical power in each of at least three equal regions of the first beam;
a second detector element that detects laser light from the second beam and outputs second signals representing optical power in each of at least three equal regions of the second beam; and
an intelligence module, coupled with the first and second detector elements and configured to receive the first and second signals and, based on the first and second signals and on distances between the at least three regions in each of the first and second beams, compute a velocity of the gas along three orthogonal directions.

14. The device of claim 13, wherein the first and second detector elements each comprise a quadrant detector that detects in four different regions.

15. The device of claim 13, wherein the first and second detector elements each comprise a separate detector for each of the at least three regions being detected.

16. The device according to claim 13, wherein the gas includes at least one of H₂O, CO₂, CH₄, N₂O, NH₃, or isotopes of CO₂ and/or H₂O.

17. The device according to claim 13, wherein the intelligence module is further configured to compute a concentration of the gas and determine a flux of the gas.

18. The device according to claim 8, wherein the intelligence module is further configured to compute a concentration of the gas and determine a flux of the gas.

19. A method of measuring a velocity of a gas, the method comprising:
expanding a first beam of laser light into a second beam having a second diameter larger than a diameter of the first beam;
detecting laser light from the second beam and outputting signals representing optical power in each of at least two regions of the second beam, wherein the gas traverses at least a portion of the second beam; and
computing, based on the signals and on a distance between the at least two regions, a velocity of the gas along at least a first direction perpendicular to the second beam.

20. The method of claim 20, wherein the gas includes at least one of H₂O, CO₂, CH₄, N₂O, or NH₃, or isotopes of CO₂ and/or H₂O.

21. The method of claim 20, further comprising computing one or both of a concentration of the gas and a flux of the gas.

22. The method of claim 20, further comprising rendering on a display device a visual representation of the velocity of the gas.
